(19) United States
(12) Patent Application Publication
(10) Pub. No.: US 2026/0369346 A1
(43) Pub. Date: Nov. 19, 2026

(54) SYSTEM AND METHOD FOR PROVIDING A DRUG DISCOVERY PLATFORM

(71) Applicant: University of Florida Research Foundation, Incorporated, Gainesville, FL (US)

(72) Inventor: David A. OSTROV, Gainesville, FL (US)

(21) Appl. No.: 19/676,038

(22) Filed: May 13, 2026

Related U.S. Application Data
(60) Provisional application No. 63/806,794, filed on May 16, 2025.

(51) Int. Cl. G16B 15/30; G06N 20/20; G16B 15/20; G16B 40/10

(52) U.S. Cl. CPC G16B 15/30; G06N 20/20; G16B 15/20; G16B 40/10

(57) ABSTRACT

Various embodiments of the present disclosure provide methods, apparatus, systems, computing devices, computing entities, and/or the like for receiving a multi-omics dataset corresponding to a set of compounds, identifying, using real-time parallel screening techniques, and based at least in part on the multi-omics dataset, a subset of target compounds from the set of compounds associated with one or more biomarkers of a biological target, generating, using one or more machine learning models, one or more predicted compound efficacies associated with the subset of target compounds and the biological target, validating, using a regulatory-ready data component, the one or more predicted compound efficacies associated with the subset of target compounds and the biological target, and initiating performance of one or more prediction based actions based on the one or more predicted compound efficacies.

### CROSS-REFERENCE TO RELATED APPLICATIONS

This application claims priority to and the benefit of U.S. Provisional Patent Application No. 63/806,794, entitled “SYSTEM AND METHOD FOR PROVIDING A DRUG DISCOVERY PLATFORM,” which was filed May 16, 2025, the entirety of which is hereby incorporated by reference.

### BACKGROUND

Various embodiments of the present disclosure address technical challenges related to identifying and optimizing drug candidates.

Drug discovery platforms are tools and technologies that help researchers accelerate the process of finding and developing new medicines. These platforms can be software-based, AI-powered applications configured to streamline components of drug discovery. For example, drug discovery platforms may enable target identification, lead optimization, and preclinical testing, for example. Drug discovery platforms may help identify potential drug targets by analyzing datasets of biological information, such as genomics, proteomics, and signaling pathways. Platforms can facilitate the identification of lead compounds by screening large libraries of molecules, physically and/or virtually, and predicting their activity against identified targets. Some platforms may provide tools for simulating pre-clinical experiments, thereby enabling researchers to predict behavior of drug candidates in vitro and in vivo.

### BRIEF SUMMARY

In general, various embodiments of the present disclosure provide methods, apparatus, systems, computing devices, computing entities, and/or the like for providing a drug-discovery platform.

In some embodiments, a computer-implemented method comprises: receiving a multi-omics dataset corresponding to a set of compounds, identifying, using real-time parallel screening techniques, and based at least in part on a multi-omics dataset, a subset of target compounds from the set of compounds associated with one or more biomarkers of a biological target, generating, using one or more machine learning models, one or more predicted compound efficacies associated with the subset of target compounds and the biological target, validating, using a regulatory-ready data component, the one or more predicted compound efficacies associated with the subset of target compounds and the biological target, and initiating performance of one or more prediction-based actions based on the one or more predicted compound efficacies.

In some embodiments, the regulatory-ready data component is configured to provide efficient hit-to-lead conversion.

In some embodiments, the computer-implemented method further comprises predicting, by the one or more processors, compound toxicity and pharmacokinetics associated with the subset of target compounds and the biological target.

In some embodiments, the real-time parallel screening techniques include phenotypic screening techniques for analyzing the set of compounds and the multi-omics dataset.

In some embodiments, the real-time parallel screening techniques include biophysical screening techniques for analyzing the set of compounds and the multi-omics dataset.

In some embodiments, the computer-implemented method further comprises enabling, by the one or more processors, de novo molecular design based, at least in part, on the one or more predicted compound efficacies and the subset of target compounds.

In some embodiments, validating the one or more predicted compound efficacies comprises verifying compliance with one or more regulatory documents.

In some embodiments, a drug discovery platform comprises: a structural analysis module configured to identify one or more druggable pockets on a target protein, an artificial intelligence (AI) module configured to (i) prioritize the one or more druggable pockets and (ii) predict one or more ligand interactions, a screening engine configured to perform virtual screening on the one or more druggable pockets simultaneously, and a validation engine configured to evaluate a set of candidate compounds to determine one or more binding affinities associated with the set of candidate compounds and the one or more druggable pockets.

In some embodiments, the structural analysis module is further configured to identify one or more cryptic pockets that are not observable in static conformations.

In some embodiments, the structural analysis module is further configured to use ensemble-based or MD-derived structures to identify the one or more cryptic pockets.

In some embodiments, the AI module is further configured to incorporate reinforcement learning to refine compound evaluation based at least in part on one or more experimental results.

In some embodiments, the structural analysis module is further configured to derive structural information from X-ray crystallography, cryo-electron microscopy, or AlphaFold-predicted models.

In some embodiments, the artificial intelligence module is further configured to predict binding affinities, pocket druggability, or conformational flexibility using a deep learning model.

In some embodiments, the validation engine is further configured to (i) determine a ranking of the one or more druggable pockets based at least in part on one or more predicted druggability values associated with the one or more druggable pockets, and (ii) select one or more compounds of the set of candidate compounds based at least in part on the ranking of the one or more druggable pockets.

In some embodiments, the drug discovery platform comprises a compound synthesis module configured to (i) synthesize the selected one or more compounds and (ii) subject the selected one or more compounds to one or more experimental assays.

In some embodiments, one or more non-transitory computer-readable storage media including instructions that, when executed by one or more processors, cause the one or more processors to perform operations comprising: receiving a multi-omics dataset corresponding to a set of compounds, identifying, using real-time parallel screening techniques, and based at least in part on a multi-omics dataset, a subset of target compounds from the set of compounds associated with one or more biomarkers of a biological target, generating, using one or more machine learning models, one or more predicted compound efficacies associated with the subset of target compounds and the biological target, validating, using a regulatory-ready data component, the one or more predicted compound efficacies associated with the subset of target compounds and the biological target, and causing execution of one or more prediction-based actions based on the one or more predicted compound efficacies.

### BRIEF DESCRIPTION OF THE DRAWINGS

FIG. 1 provides an example overview of a framework in accordance with some embodiments of the present disclosure.

FIG. 2 provides an example computing entity in accordance with some embodiments of the present disclosure.

FIG. 3 provides an example user computing entity in accordance with some embodiments of the present disclosure.

FIG. 4 depicts an example conservation of IEDB prediction tool functions in a subject web application in accordance with at least some embodiments of the present disclosure.

FIG. 5 depicts an example automated graphing 500 of T cell epitope predictions in accordance with at least some embodiments of the present invention.

FIG. 6 depicts an example series of panels depicting identification of immunodominant epitopes in candidate autoantigens in accordance with at least some embodiments of the present invention.

FIG. 6 is a flowchart diagram of an example process for determining parameter values of the electro-stimulation device on a subject to achieve improved cognitive function, in accordance with at least some example embodiments of the present invention.

FIG. 7. depicts an example series of panels depicting identification of immunodominant epitopes in MART-1 in accordance with at least some embodiments of the present invention.

FIG. 8 depicts an example series of panels depicting identification of high-affinity T cell epitopes from SARS-CoV-2 spike protein in accordance with at least some embodiments of the present invention.

FIG. 9 is a flowchart depicting an example process for identifying and optimizing drug candidates in accordance with at least some embodiments of the present invention.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout.

Embodiments of the present disclosure include systems, methods, computer program products, and techniques for identifying and optimizing drug candidates.

### Overview, Technical Improvements, and Technical Advantages

Various embodiments of the present disclosure make important technical contributions to fields and/or environments for identifying and optimizing drug candidates.

Embodiments of the present disclosure provide a drug discovery platform combining cutting-edge automation, artificial intelligence (AI), and advanced assay technologies to revolutionize the process of identifying and optimizing drug candidates. The drug discovery platform integrates robotic systems, parallel screening capabilities, and real-time data processing to enable the efficient testing of thousands of compounds across multiple biological targets simultaneously. AI and machine learning enhance each step, from identifying novel or “undruggable” targets to predictive modeling of compound efficacy, toxicity, and pharmacokinetics, as well as de novo molecular design. The platform features advanced phenotypic and biophysical screening methods, including organoids and 3D cultures, and leverages multi-omics data for comprehensive target identification. With support for both small molecule and biologic discovery, emerging modalities like RNA-based drugs and PROTACs, and innovative chemistry tools such as structure- and fragment-based drug design, it delivers unprecedented flexibility and scalability. A robust in silico infrastructure ensures cost-effective operations with virtual screening, molecular dynamics simulations, and cloud computing. The platform is designed for scalability across therapeutic areas, from oncology to CNS diseases, and integrates biomarker discovery to guide precision medicine approaches. Validated output, customizable workflows, and regulatory-ready data components ensure efficient hit-to-lead conversion, preclinical validation, and reduced timelines, thereby accelerating drug development. In general, embodiments of the present disclosure enhance efficiency, scalability, and ability with respect to producing high-quality, innovative therapeutic candidates.

### Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### Example Framework

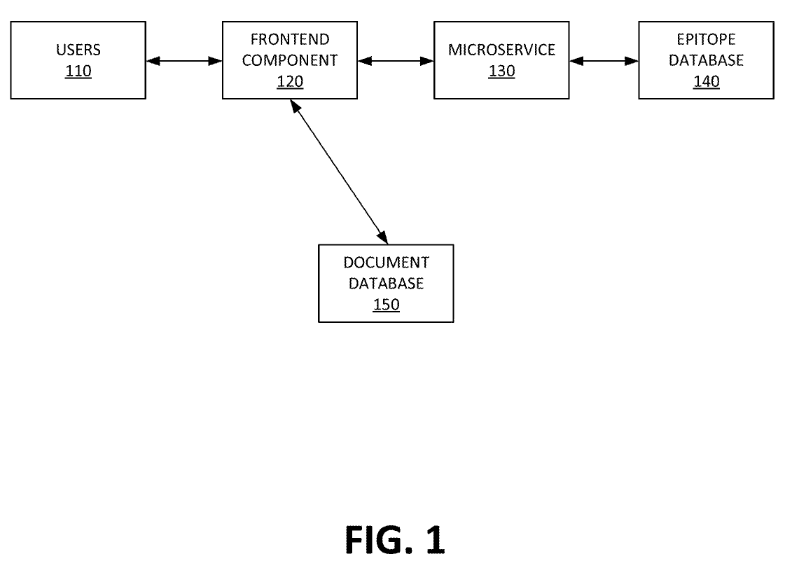

FIG. 1 provides an example overview of a framework 100 in accordance with some embodiments of the present disclosure. The framework 100 may correspond to a system for drug discovery. As depicted, framework 100 includes frontend component 120, microservice 130, epitope database 140, and document database 150. Framework 100 may provide a drug discovery platform combining cutting-edge automation, AI, and advanced assay technologies to improve the process of identifying and optimizing drug candidates.

Frontend component 120 may be a computing device comprising a web application interface enabling access to a plurality of additional components, such as microservice 130, epitope database 140, and document database 150. In at least some embodiments, frontend component 120 includes a front-end graphical user interface, enabling one or more users 110 to interact with the various additional components.

In some embodiments, frontend component 120 is configured to provide a dynamic and responsive user interface for accessing epitope prediction and analysis functionalities. The frontend component 120 may be developed using React and TypeScript, enabling a graphical user interface (GUI) that facilitates user interaction with the underlying microservices and databases. The frontend component 120 may be configured to enable access to the microservice 130, the epitope database 140, and the document database 150.

The frontend component 120 may be communicatively coupled to the microservice 130, which features an application programming interface (API) integrated with epitope prediction tools. The microservice 130 may be configured to interface with the Immune Epitope Database (IEDB), enabling users to submit queries directly to the IEDB prediction tools via the application. The functionality of the native IEDB tools is retained within the framework 100, allowing users to submit queries to the IEDB server and review prediction data within a single unified environment.

The frontend component 120 may be communicatively coupled to the epitope database 140, which comprises the Immune Epitope Database (IEDB). The framework 100 may be configured to fetch prediction data from the epitope database 140, enabling users to submit queries to MHC Class I and Class II T cell epitope prediction tools and receive data including immunodominant peptide sequences and binding affinity predictions.

The frontend component 120 may be communicatively coupled to the document database 150. The document database 150 provides user services including a workspace for saving, recalling, and editing searches. The backend infrastructure ensures robust data handling by managing data submissions, retrievals, and user accounts.

The frontend component 120 may be configured to provide a plurality of interactive features for data visualization and analysis. The users 110 may be enabled to interact with graphs that support zooming, display of data point details upon selection, and adjustable scales including logarithmic and linear representations. In some embodiments, the frontend component 120 further provides customizable graph features including line thickness adjustment and y-axis range configuration. The application supports overlaying multiple datasets, filtering data based on user-defined criteria, and downloading both visualizations and raw data in formats including images and CSV files, thereby facilitating the generation of publication-quality figures within the unified environment.

Microservice 130 may include an application built as a collection of small, autonomous, and independent services, wherein each service is designed to perform a specific function and communicate with other services through lightweight protocols such as REST APIs or message queues. In at least some embodiments, microservice 130 incorporates a modular approach enabling faster development cycles, improved scalability, and increased resilience. In at least some embodiments, microservices 130 may comprise a collection of independent services, each potentially running on a different server, for example. Microservices 130 may be loosely coupled and autonomous.

In some embodiments, the microservice 130 features an application programming interface (API) integrated with epitope prediction tools, enabling the users 110 to submit queries directly to the IEDB prediction tools via the application while retaining the full functionality of the native IEDB tools.

The microservice 130 may comprise an epitope prediction service configured to allow users to submit queries to MHC Class I and Class II T cell epitope prediction tools. In some embodiments, the epitope prediction service is configured to receive and process data including immunodominant peptide sequences and binding affinity predictions. The users 110 can submit queries to an IEDB server and review IEDB data within a single environment. In some embodiments, a resultant IEDB-generated data file can be reviewed in database format and downloaded by the users. Native IEDB outputs may identify candidates by binding rank and predicted score, and these parameters are retained within the framework 100. The epitope prediction service may be configured to back-calculate the affinity, expressed as a dissociation constant value (Kd), from the IEDB reported score.

The microservice 130 may further comprise a data retrieval service configured to communicate with the Immune Epitope Database to fetch prediction data. The data retrieval service enables complex data management and user preference storage, supporting a seamless user experience within the unified environment.

In some embodiments, the microservice 130 comprises a visualization service configured to allow the users 110 to automatically plot prediction data and visually analyze data using customizable graphs. The visualization service may remove the need to replot data offline and generate overlays, shifting analysis to a visual modality. The visualization service may be configured to enable the users 110 to identify core sequences and their positions relative to the queried protein sequence.

In some embodiments, the microservice 130 further comprises a user account service configured to manage data submissions, retrievals, and user accounts. The user account service provides a workspace for saving, recalling, and editing searches. In some embodiments, the microservice 130 provides a database access feature enabling review of text and numerical data consistent with browser-based and downloadable CSV files from the epitope database 140.

In some embodiments, the microservice 130 additionally comprises a binding affinity classification service configured to enable scaling that allows the users 110 to generate and analyze on predicted affinity constants (Kd values). The binding affinity classification service is configured to identify high affinity (< 50 nM), moderate affinity (50 – 500 nM), and low affinity (> 5,000 nM) binding T cell epitope candidates.

In some embodiments, the microservice 130 further comprises a data export service configured to support overlaying multiple datasets, filtering data based on user-defined criteria, and downloading both visualizations and raw data in formats including images and CSV files. The data export service may facilitate the generation of publication-quality figures within the unified environment.

Epitope database 140 may be a comprehensive database and analysis resource configured to store and/or provide experimentally measured and predicted immunological data, particularly focusing on epitopes. In at least some embodiments, epitope database 140 curates and compiles experimental data from scientific literature and user submissions. Epitope database 140 may be configured to include data on both antibody and T cell epitopes, covering a wide range of diseases. In at least some embodiments, epitope database 140 may comprise a suite of tools for predicting and analyzing epitopes and may provide valuable insights in various fields including vaccine development, diagnostics, and therapeutics. IEDB.org is an example of an appropriate epitope database 140.

In some embodiments, the framework 100 comprises an epitope database 140 communicatively coupled to the microservice 130. The epitope database 140 may comprise the IEDB accessible at IEDB.org, a publicly accessible repository funded by the National Institute of Allergy and Infectious Disease (NIAID). The epitope database 140 serves as a comprehensive data source for experimental data regarding antibody and T cell epitopes across multiple disciplines.

The epitope database 140 may be configured to store and provide access to experimental data spanning multiple immunological disciplines including allergy, autoimmunity, histocompatibility, cancer, and infectious disease. The repository contains curated data regarding peptide-HLA interactions, binding affinities, and immunogenicity measurements derived from experimental studies.

In some embodiments, the epitope database 140 further comprises T cell epitope prediction tools configured to analyze protein sequences of interest presented by individual major histocompatibility complex (MHC) allotypes. The prediction tools may be configured to generate predictions for binding affinity, processing, and immunogenicity of peptide epitopes. The epitope database 140 supports both MHC Class I and MHC Class II binding predictions, enabling comprehensive analysis of T cell epitope candidates.

In some embodiments, the epitope database 140 is configured to identify immunodominant epitopes from user-supplied protein sequences. The prediction tools generate ranked lists of predicted binding core sequences and scores based on predicted binding affinity. The epitope database 140 outputs downloadable text and numerical data including binding rank, predicted score, and peptide sequence information.

The microservice 130 may be configured to communicate with the epitope database 140 to fetch prediction data, enabling seamless query submission and data retrieval within the unified environment. The data retrieval service of the microservice 130 may interface with the epitope database 140 to obtain prediction results, which are then processed to back-calculate the dissociation constant (Kd) values from the reported scores. This integration enables users to submit queries directly to the epitope database 140 via the microservice 130 and receive comprehensive binding affinity data within the frontend component 120.

Document database 150 may be a source-available, cross-platform, document-oriented database program. In at least some embodiments, document database 150 is configured as a NoSQL database product. Document database 150 may be configured to use JSON-like documents with optional schemas. In at least some embodiments, document database 150 may support features like sharding, replication, and ACID transactions.

In some embodiments, the document database 150 is implemented using MongoDB and is configured to provide persistent data storage for user-generated content and query results within the system architecture. The document database 150 may be configured to store user credentials, authentication data, and user preferences, enabling secure access to the system and personalized user experiences. The document database 150 supports user account management functions including registration, login authentication, and maintenance of user-specific settings and configurations.

In some embodiments, the document database 150 provides a workspace functionality configured to enable saving, recalling, and editing of searches. The users 110 may organize and manage multiple queries over time, with the document database 150 maintaining persistent records of user-defined search parameters and configurations. This workspace functionality enables the users 110 to return to previous analyses without re-entering query parameters.

The document database 150 may be configured to store text and numerical data consistent with browser-based and downloadable CSV files from the epitope database 140. The stored data includes prediction results, binding ranks, scores, and calculated dissociation constant (Kd) values derived from epitope prediction queries. This data storage capability enables the users 110 to review and compare results from multiple prediction analyses within the unified environment.

In some embodiments, the document database 150 maintains records of submitted queries and associated results, enabling the users 110 to recall previous analyses without resubmitting queries to the epitope database 140. This query history functionality reduces redundant data requests and enables efficient longitudinal analysis of epitope prediction data across multiple sessions.

A backend infrastructure (powered by Java Spring Framework, for example) interfaces with the document database 150 to manage data submissions, retrievals, and user accounts. This integration ensures robust data handling and supports the seamless user experience by enabling complex data management operations and user preference storage within the system architecture.

In at least some embodiments, framework 100 is configured to provide high-throughput capabilities to accelerate drug discovery and development. Framework 100 may include robotic systems for large-scale compound screening, synthesis, or cellular assays. In at least some embodiments, framework 100 leverages parallel screening techniques to test thousands of molecules across multiple biological targets simultaneously. Framework 100 may be configured for real-time data collection and processing to identify promising candidates quickly. In at least some embodiments, framework 100 is configured to leverage Ultra-Performance Liquid Chromatography systems with Photodiode Array (UPLC-PDA) and Quadrupole Detector Array (QDA), UPLC coupled with Tandem Mass Spectrometry (UPLC-MS/MS), and Ultra-High-Performance Liquid Chromatography Quadrupole Time-of-Flight (UHPLC-Q-TOF) systems. These instruments enable precise and efficient analysis of complex compounds, facilitating large-scale screening and synthesis. In at least some embodiments, the framework 100 is configured to utilize advanced robotic systems and microtiter plates to translate benchtop assays into high-density formats suitable for large-scale screening, thereby accelerating the identification of potential therapeutic leads by allowing rapid assessment of compound libraries against diverse pharmacological targets.

In at least some embodiments, framework 100 is configured to leverage Radiomics and Augmented Intelligence Laboratory (RAIL) techniques. Radiomics-based AI models include AI tools that extract and analyze quantitative features from medical images to identify biomarkers for a range of diseases. Machine learning models developed through this technology predict disease outcomes, particularly in areas such as head and neck imaging. These tools enable precise tumor characterization and treatment response evaluation, supporting personalized therapeutic strategies.

In at least some embodiments, framework 100 is configured to integrate AI tools into clinical workflows to ensure diagnostic accuracy, streamline healthcare processes, and improve patient outcomes by ensuring the solutions as described herein are accessible and practical for clinical use. Further, framework 100 is configured to foster interdisciplinary collaboration to advance development and validation of AI technologies.

In at least some embodiments, framework 100 is configured to leverage a cloud-based image analysis platform that integrates advanced deep neural network (DNN)-based AI technologies to automate the analysis of high-resolution whole-slide images (WSIs) of histologically stained tissue sections. Such image analysis platforms offer an end-to-end solution combining massive scalability, on-demand cloud computing, and an intuitive web-based user interface via which users can readily upload, visualize, and interact with data, thereby facilitating the integration of computational pathology into routine diagnostics and research. In at least some embodiments, framework 100 is equipped with AI algorithms for image analysis to identify clinically relevant micro-anatomic functional tissue units and to extract image features.

In at least some embodiments, framework 100 is configured to leverage Radiology Pathology Image Alignment (RAPHIA) deep learning pipelines to facilitate the precise registration of magnetic resonance imaging (MRI) with whole-mount histopathology images of the prostate. This alignment enables accurate mapping of the extent of prostate cancer, for example, thereby enhancing the development of machine learning models for early detection. RAPHIA automates several traditionally manual and time-consuming steps, including prostate segmentation, estimation of rotation angles, and horizontal flipping in histopathology images, and determination of MRI-histopathology slice correspondences. By employing deep learning registration networks, RAPHIA significantly reduces computational time and improves accuracy. Notably, it eliminates the need for multimodal image similarity metrics by transforming histopathology image representations to MRI image representations and vice versa. This innovative approach enables even novice users to achieve expert-level performance in image registration tasks, thereby streamlining the processing of large datasets useful for developing robust machine learning models for prostate cancer detection on MRI.

In at least some embodiments, framework 100 is configured to leverage scout Electroencephalography Source Imaging (ESI) and Convolutional Neural Network (CNN) techniques, configured to combine ESI with CNN techniques to classify motor imagery tasks, thereby enhancing the decoding of neural signs associated with motor functions, which is useful for developing brain-computer interfaces (BCIs) and neuroprosthetics. By accurately interpreting motor imagery through EEG, researchers can better understand the neural underpinnings of motor control and dysfunction.

In at least some embodiments, framework 100 is configured to leverage natural language processing (NLP) techniques to analyze patient records and advance directives to guide surrogate decision-makers in line with patient goals. Machine learning models are instrumental in predicting the efficacy and safety of drugs, significantly reducing the time and costs associated with clinical trials. Framework 100 may be configured to leverage AI models to enhance treatment optimization by helping clinicians choose the best treatment options. As such, framework 100 uses patient data, clinical guidelines, and real-world evidence to make informed decisions. For example, in radiation oncology, AI optimizes radiation dose distribution to minimize damage to healthy tissue(s). In the realm of surrogate and proxy decision-making, AI models may be configured to incorporate patient preferences and values into medical recommendations, thus supporting ethical decision-making for incapacitated patients. Natural language processing techniques may be used to analyze patient records and advance directives, providing guidance to surrogate decision-makers that aligns with the patients’ goals.

In at least some embodiments, framework 100 is configured to automate routine tasks such as generating radiology reports, interpreting lab results, and managing triage in emergency settings. Clinical support systems leverage patient data and integrate it with evidence-based knowledge to assist in real-time decision-making at the point of care.

In at least some embodiments, framework 100 is configured to leverage a miROMICs platform configured to employ machine learning and advanced bioinformatics to analyze microRNA (miRNA) interactions and their impact on diseases, which can be pivotal in drug discovery and development. By predicting and profiling the interactions between miRNAs and their targets, miROMICs offers insights into potential therapeutic targets and biomarkers for various diseases, thereby streamlining the development of drugs by identifying key molecular pathways involved in disease progression and enhancing the efficiency and specificity of therapeutic interventions.

In at least some embodiments, framework 100 is configured to leverage a web-based tool developed for integrating spatial -OMICS technologies with histopathological analysis and enabling comprehensive analysis of molecular and histopathologic data from Whole Slide Images (WSIs). By leveraging deep learning algorithms and a suite of analytical tools, framework 100 facilitates end-to-end analysis of functional tissue units, thereby correlating molecular data with histopathology. This allows for the identification of pathomic features that provide insights into both healthy and altered cellular states, thereby enhancing the understanding of disease mechanisms and potentially guiding development. Through techniques such as 10x Visium spatial transcriptomics, framework 100 demonstrates its capability to dissect complex tissue environments, thereby bridging qualitative and quantitative data for robust biomarker and target discovery.

In at least some embodiments, framework 100 is configured to leverage advanced assay technologies such as phenotypic screening, biophysical methods, and organoids and 3D cultures. Phenotypic screening may include using functional assays to identify compounds that affect cellular or disease phenotypes. Biophysical methods include using techniques like X-ray crystallography, cryo-EM, or SPR (surface plasmon resonance) for target validation and hit optimization. Organoids and 3D cultures use advanced cell culture models to mimic in vivo conditions. Phenotypic screening spans a broad spectrum of both common and rare diseases, thereby enabling a comprehensive approach to drug discovery.

In at least some embodiments, framework 100 is configured to leverage sophisticated biophysical methods such as x-ray crystallography, cryo-electron microscopy, and surface plasmon resonance (SPR). These techniques are useful for target validation and hit optimization during the drug development process. X-ray crystallography and cryo-EM allow for detailed structural analysis of drug targets, thereby providing insights into the molecular interactions and dynamics useful for effective drug design. SPR complements these methods by offering real-time measurement of the binding kinetics and affinity between drugs and their targets. In at least some embodiments, framework 100 is configured to facilitate the study of host-pathogen interactions, such as how pathogens infect human cells.

Framework 100 may be configured to integrate multiple omics datasets for comprehensive target identification. By leveraging advanced genomic technologies, framework 100 may identify genetic variations and mutations associated with diseases, thereby providing targets for novel drug therapies. Transcriptomic analyses enhance understanding of disease by charting changes in gene expression across different conditions and responses to treatments, enabling the identification of biomarkers and therapeutic targets.

In at least some embodiments, framework 100 is configured to employ mass spectrometry and other high-throughput technologies to analyze protein expressions and interactions, which are useful for understanding disease mechanisms at a molecular level and for validating drug targets. The integration of these omics technologies facilitates a comprehensive approach to drug discovery, supporting initial discovery of drug targets through the optimization and validation of drug candidates. Multi-omics integration speeds up the drug discovery process while also increasing its accuracy, thereby providing more effective and personalized therapeutic solutions. In at least some embodiments, framework 100 is configured with one or more AI-driven tools designed to analyze complex genetic datasets (such as genetics, single-cell genomics, and metagenomics) using deep learning and statistical methods.

Framework 100 may be configured for single-cell analysis techniques, including employing high-throughput sequencing services that enable detailed genetic profiling of individual cells. Such genetic profiling is instrumental in identifying unique genetic markers and understanding disease mechanisms at an unprecedented resolution. Further, this capability not only bolsters the development of treatments tailored to individual genetic profiles, but also significantly accelerates the path from research to clinical application by ensuring potential therapies are effective against intended targets within complex tissue environments.

In at least some embodiments, framework 100 is configured to leverage multiple specialized cores and resources for the discovery of both small molecules and biologics such as antibodies, peptides, and proteins. Framework 100 may be configured to develop gene therapies for genetic disorders such as cystic fibrosis and alpha 1-antitrypsin deficiency, for example. Such gene therapies may include using adeno-associated virus (AAV) vectors to safely and effectively deliver therapeutic genes to target tissues. In at least some embodiments, framework 100 is configured to maintain a library of AAV reporter vectors.

Framework 100 may be configured for structure-based drug design (SBDD), leveraging structural biology for rational drug design, or for fragment-based drug discovery (FBDD), screening smaller molecular fragments for high-affinity binding. In at least some embodiments, framework 100 is configured for “Machine Gun Docking” (or MGD), which utilizes multiple structural pockets to identify and select potential lead compounds. This approach targets distinct structural regions on key proteins to identify new inhibitors and can be adapted to target a variety of proteins associated with different diseases.

In an example embodiment, MGD techniques are used to explore four unique structural pockets in an antigen-binding cleft of an HLA-DQ8 molecule. This effort results in the discovery of active inhibitors which could prevent T cell recognition, underscoring the potential of structure-based drug design to bridge the gap from computational models to clinical applications and illustrating a direct pathway from the virtual environment of drug design to real-world clinical settings.

In at least some embodiments, framework 100 is configured to utilize dynamic properties to enhance the identification of true binders and non-binders in protein-ligand interactions to determine a “DyScore.” When determining the DyScore, framework 100 may be configured to incorporate a nuanced consideration of the dynamic nature of complex molecular interactions, thereby limiting inaccuracies suffered by traditional scoring functions. By providing a robust method for evaluating potential effectiveness of molecules, DyScore helps streamline the development pipeline, reducing time and costs associated with experimental assays and increasing the likelihood of success in clinical phases.

Framework 100 may be configured to leverage Fragment-Based Drug Discovery (FBDD) techniques configured to screen small molecular fragments for their ability to bind with high affinity to target proteins. FBDD techniques facilitate efficient identification and optimization of lead compounds that can be developed into full-sized drugs. FBDD techniques, as leveraged by framework 100, are further enhanced by access to extensive chemical libraries, which may include proprietary and diverse collections of novel and synthesizable compounds. Such libraries support the iterative process of FBDD, where fragments demonstrating promising binding properties are elaborated into more potent molecules through various chemical modifications. The combination of FBDD expertise and chemical libraries provides a dynamic platform for drug discovery, thereby enhancing the likelihood of discovering viable drug candidates with optimal pharmacological profiles.

In at least some embodiments, framework 100 is configured to leverage virtual screening techniques for computational docking to prioritize compounds for synthesis or testing. Framework 100 may be configured to predict binding affinity and stability via molecular dynamics simulations and modeling. In at least some embodiments, framework 100 is configured to leverage cloud computing mechanisms for handling extensive computational demands associated with big data analysis and modeling in drug discovery by utilizing scalable computational resources to process large datasets. Notably, utilization of cloud services enables computational resources to be dynamically scaled to meet the needs of various projects, thereby improving efficiency and reducing time from initial research to potential drug candidates.

Framework 100 may be configurable for target class flexibility by supporting G protein-coupled receptors (GPCRs), kinases, ion channels, and other challenging target classes. For example, framework 100 may enable the use of small molecules to inhibit antigen presentation as a strategy to prevent autoimmune diseases, long-acting versions of existing drugs for managing Type 1 diabetes, and targeted cancer therapies using PKC inhibitors for ovarian cancer. Framework 100 may additionally be configured for the identification of sigma receptor ligands, exploration of fibroblast growth factor 7 peptides (FGF7-P) for therapeutic applications, and efforts in COVID-19 treatment and prevention strategies, reflecting its role in rapid response to emerging health crises.

Framework 100 may be configured to integrate with external tools, including industry-standard software and databases. For example, framework 100 may be configured to interface with comprehensive chemical and biological databases to enhance the scope of target validation and drug optimization studies, thereby allowing researchers to access vast repositories of existing data on compound structures, biological activities, and pharmacological properties. Compatibility with advanced modeling and simulation software facilitates detailed study of drug-target interactions at the molecular level, thereby optimizing binding affinity and selectivity. This integration enables streamlined workflows and improved efficiency by leveraging cutting-edge computational tools for structure-based drug design, quantitative structure-activity relationship (QSAR) modeling, and virtual screening.

In at least some embodiments, framework 100 is configured to tailor workflows for specific therapeutic areas or partners. For instance, whether the focus is on oncology, neurodegenerative diseases, or infectious diseases, the platform can adjust its computational models, screening libraries, and validation processes accordingly. This adaptability is particularly valuable when collaborating with external partners; by customizing the drug discovery process, the platform ensures that it aligns with the partner’s objectives, thereby facilitating a more target approach to compound screening and optimization.

Framework 100 may be configured to guide patient selection or track therapeutic responsive based on the identification of biomarkers. For example, framework 100 may be configured to incorporate advanced methodologies for the identification of predictive biomarkers using functional Magnetic Resonance Imaging (fMRI) to explore neurological biomarkers. This method is particularly effective in understanding brain activity in real-time, which can be pivotal in developing targeted therapies for neurological disorders. In at least some embodiments, framework 100 is additionally configured to leverage a variety of other biomarker discovery techniques. Genomic and proteomic profiling may be employed to identify molecular signatures that can predict drug efficacy or the likelihood of adverse reactions to treatments.

In at least some embodiments, framework 100 is configured to leverage translational research techniques to use biomarkers to connect preclinical models with clinical applications. Innovative projects exemplify this approach across various diseases; for example, there exist biomarkers for Alzheimer’s disease that help in identifying early stages of the disease by detecting specific proteins or genetic markers, which can be targeted for early therapeutic intervention. Further extending the reach of translational research, the discovery of cancer biomarkers involves identifying genetic expressions and mutations specific to various cancer types. These biomarkers facilitate targeted therapy regimes in clinical trials, improving the precision of treatments tailored to individual genetic profiles. Such discoveries not only expedite the process from lab research to practical treatment options but also enhance the efficacy and personalization of cancer therapies.

Framework 100 may be configured to implement a centralized/unified database for tracking compounds, targets, and assay results. Such a system streamlines data accessibility and facilitates the integration of new data management technologies, which are useful for improving the efficiency and accuracy of drug discovery efforts. By centralizing data, researchers can leverage advanced analytics to identify subtle patterns and relationships that might be overlooked in more fragmented systems. This centralized approach not only accelerates the discovery and evaluation of new drug candidates by providing a comprehensive view of all experimental data but also enhances the ability to predict how different compounds will interact with various targets. Integrating such analytical techniques within a centralized data framework significantly propels the drug discovery process forward, making it more robust and responsive to new scientific insights.

Incorporating real-time analytics into framework 100 profoundly enhances the decision-making process by providing researchers with dynamic dashboards that monitor platform performance and other metrics. These dashboards offer immediate insights into vast datasets, allowing for the agile adjustment of experiments and strategies based on current data trends. Utilizing real-time analytics, researchers can observe interaction dynamics between novel drug candidates and their targets as they happen, enabling the immediate evaluation of compound efficacy and safety. Real-time analytics support a more nuanced and rapid response to experimental data, accelerating the path from discovery to development, and ultimately, to market.

Framework 100 may be configured to maintain compliance with stringent data and research standards, which may be useful for regulatory readiness. This may include comprehensive documentation processes that adhere to the guidelines set forth by regulatory bodies such as the FDA. A key component of framework 100 is integrated Institutional Review Board (IRB) protocols, which ensure that all research involving human subjects meets ethical and legal requirements. This regulatory-ready framework facilitates a smoother transition from preclinical development to clinical trials while upholding the integrity of the research. Having a well-established IRB process in place significantly ensures that all experimental data, particularly data involving human subjects, is collected, stored, and used in compliance with regulatory standards. This not only minimizes potential legal and ethical risks but also builds trust with stakeholders, including participants, regulatory agencies, and potential commercial partners. By streamlining compliance and documentation processes, the platform can accelerate its timelines for drug development, moving more efficiently from discovery to market while adhering to the highest standards of research integrity and safety.

In at least some embodiments, framework 100 is a drug discovery platform comprising a plurality of modules. In some embodiments, the drug discovery platform comprises a structural analysis module configured to identify one or more druggable pockets on a target protein. The structural analysis module may be further configured to identify one or more cryptic pockets that are not observable in static conformations. In some embodiments, the structural analysis module is further configured to use ensemble-based or MD-derived structures to identify the one or more cryptic pockets. In some embodiments, the structural analysis module is further configured to derive structural information from X-ray crystallography, cryo-electron microscopy, or AlphaFold-predicted models.

The drug discovery platform may additionally comprise an artificial intelligence (AI) module configured to (i) prioritize the one or more druggable pockets and (ii) predict one or more ligand interactions. In some embodiments, the drug discovery platform is further configured to predict binding affinities, pocket druggability, or conformational flexibility using a deep learning model. In some embodiments, the AI module is further configured to incorporate reinforcement learning to refine compound evaluation based at least in part on one or more experimental results.

In at least some embodiments, the drug discovery platform comprises a screening engine configured to perform virtual screening on the one or more druggable pockets simultaneously. The drug discovery platform may comprise a validation engine configured to evaluate a set of candidate compounds to determine one or more binding affinities associated with the set of candidate compounds and the one or more druggable pockets. In some embodiments, the validation engine is further configured to (i) determine a ranking of the one or more druggable pockets based at least in part on one or more predicted druggability values associated with the one or more druggable pockets, and (ii) select one or more compounds of the set of candidate compounds based at least in part on the ranking of the one or more druggable pockets.

In some embodiments, the screening engine is configured to process user-submitted protein sequences and identify immunodominant peptide epitopes through computational analysis of peptide-HLA interactions. The screening engine may be communicatively coupled to the frontend component 120, the microservice 130, and the epitope database 140, and may be configured to perform automated epitope prediction and binding affinity analysis.

The screening engine may comprise a query processing module configured to receive protein sequences from the frontend component 120 and format queries for submission to the epitope database 140 via the microservice 130. The query processing module supports both MHC Class I and MHC Class II binding predictions, enabling comprehensive analysis of T cell epitope candidates across multiple HLA allotypes.

The screening engine may further comprise a prediction algorithm interface configured to interface with T cell epitope prediction tools including NetMHCIIpan for binding affinity, processing, and immunogenicity predictions. The prediction algorithm interface retains the full functionality of native IEDB prediction methods while enabling seamless integration within the unified environment of the system.

The screening engine may additionally comprise a binding affinity calculation module configured to process IEDB reported scores to generate binding rank and predicted score data. The binding affinity calculation module is further configured to back-calculate dissociation constant (Kd) values from the reported scores to express binding affinity in standardized units, enabling direct comparison of binding strengths across different peptide-HLA combinations.

The screening engine may comprise an epitope ranking module configured to generate ranked lists of predicted binding core sequences based on binding affinity. The epitope ranking module classifies epitopes according to binding affinity thresholds, identifying high affinity, moderate affinity, and low affinity binding T cell epitope candidates.

In some embodiments, the screening engine additionally comprises a multi-allotype analysis module configured to enable simultaneous screening of protein sequences against multiple HLA allotypes. The multi-allotype analysis module supports comparative analysis across different MHC Class I and MHC Class II molecules, enabling identification of peptide epitopes that bind to multiple common HLA allotypes within a single analysis session.

The screening engine may comprise an output generation module configured to format prediction results for visualization by the frontend component 120. The output generation module may enable overlay of multiple datasets and generation of publication-quality figures, facilitating the identification of immunodominant epitopes in context of multiple specific HLA allotypes through color coding and interactive graphical representations.

In some embodiments, the drug discovery platform comprises a compound synthesis module configured to (i) synthesize the selected one or more compounds and (ii) subject the selected one or more compounds to one or more experimental assays. The compound synthesis module may be communicatively coupled to the screening engine and the document database 150, enabling automated synthesis and experimental validation of candidate compounds within the framework 100.

The compound synthesis module may comprise a synthesis subsystem configured to perform large-scale compound synthesis utilizing robotic systems for automated synthesis operations. The synthesis subsystem has access to extensive chemical libraries including proprietary and diverse collections of novel and synthesizable compounds. The synthesis subsystem may support both small molecule synthesis and biologic synthesis including antibodies, peptides, and proteins, enabling comprehensive compound generation across multiple therapeutic modalities.

The compound synthesis module may further comprise a high-throughput screening subsystem configured to enable parallel screening of synthesized compounds across multiple biological targets simultaneously. The high-throughput screening subsystem may utilize advanced robotic systems and microtiter plates with 1,536 wells for high-density screening formats. This parallel screening technology enables the simultaneous testing of thousands of molecules, significantly enhancing the scope and speed of compound validation.

In some embodiments, the compound synthesis module additionally comprises a phenotypic screening subsystem configured to perform functional assays that identify compounds affecting cellular or disease phenotypes. The phenotypic screening subsystem may utilize organoids and 3D cultures to mimic in vivo conditions, providing more relevant human tissue responses for compound evaluation. This capability enables the identification of compounds with therapeutic potential across a broad spectrum of disease states.

The compound synthesis module may further comprise a biophysical validation subsystem configured to perform target validation and hit optimization using techniques including X-ray crystallography, cryo-electron microscopy (cryo-EM), and surface plasmon resonance (SPR). The biophysical validation subsystem may enable detailed structural analysis of drug-target interactions, providing insights into molecular interactions and dynamics useful for effective drug design. SPR measurements provide real-time binding kinetics and affinity data between compounds and their targets.

In some embodiments, the compound synthesis module additionally comprises an analytical instrumentation subsystem configured to perform precise compound analysis. The analytical instrumentation subsystem includes Ultra-Performance Liquid Chromatography (UPLC) systems with Photodiode Array (PDA) and Quadrupole Detector Array (QDA), UPLC coupled with Tandem Mass Spectrometry (UPLC-MS/MS), and Ultra-High-Performance Liquid Chromatography Quadrupole Time-of-Flight (UHPLC-Q-TOF) systems. These instruments enable precise and efficient analysis of complex compounds, facilitating large-scale screening and synthesis validation.

The compound synthesis module may further comprise a hit-to-lead conversion subsystem configured to perform rapid optimization of pharmacokinetics, pharmacodynamics, and drug-like properties of identified hit compounds. The hit-to-lead conversion subsystem may be configured to support preclinical validation with high success rates in animal models, enabling efficient transition from initial hits to lead compounds ready for further development.

In some embodiments, the compound synthesis module additionally comprises a data integration subsystem configured to perform real-time data collection and processing from all experimental assays. The data integration subsystem interfaces with the document database 150 to maintain a unified database for tracking compounds, targets, and assay results. This data centralization enables seamless collection, processing, and analysis of experimental data, facilitating the rapid identification of promising candidates for further development.

### A. Example User Device

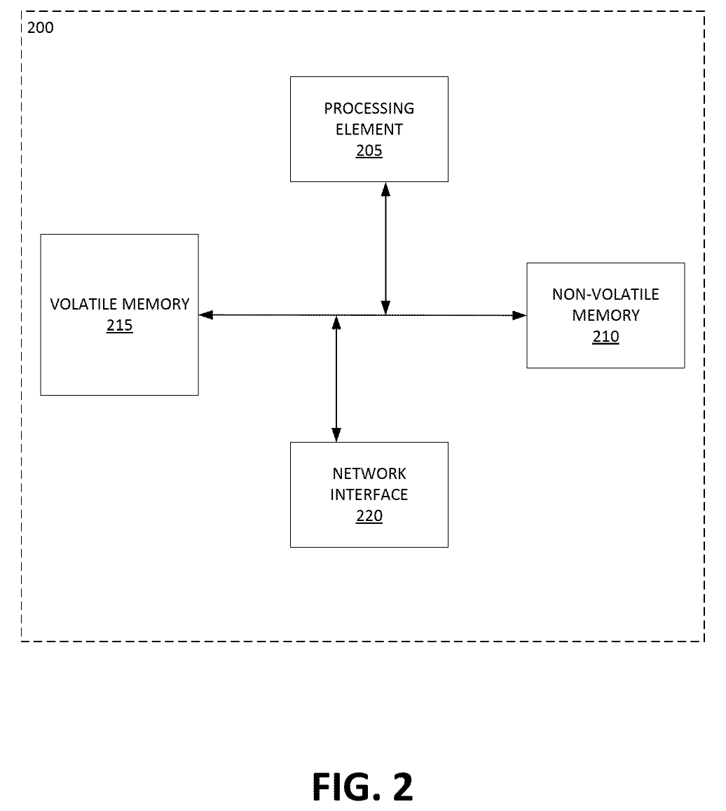

FIG. 2 provides an example computing entity 200 in accordance with some embodiments of the present disclosure. The computing entity 200 may be an example of the frontend component 120 of FIG. 1, for example. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein.

As shown in FIG. 2, in some embodiments, the computing entity 200 may include, or be in communication with, one or more processing elements 205 (also referred to as processors, processing circuitry, and/or similar terms used herein interchangeably) that communicate with other elements within the computing entity 200 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In some embodiments, the computing entity 200 may further include, or be in communication with, non-volatile media (also referred to as non-volatile storage, memory, memory storage, memory circuitry, and/or similar terms used herein interchangeably). In some embodiments, the non-volatile media may include one or more non-volatile memory 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile media may store databases, database instances, database management systems, data, applications, programs, program modules, scripts, code (e.g., source code, object code, byte code, compiled code, interpreted code, machine code, etc.) that embodies one or more computer functions described herein, executable instructions, and/or the like. The term database, database instance, database management system, and/or similar terms used herein interchangeably, may refer to a collection of records or data that is stored in a computer-readable storage medium using one or more database models; such as a hierarchical database model, network model, relational model, entity-relationship model, object model, document model, semantic model, graph model, and/or the like.

In some embodiments, the computing entity 200 may further include, or be in communication with, volatile media (also referred to as volatile storage, memory, memory storage, memory circuitry, and/or similar terms used herein interchangeably). In some embodiments, the volatile media may also include one or more volatile memory 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, code (source code, object code, byte code, compiled code, interpreted code, machine code) that embodies one or more other computer functions described herein, executable instructions, and/or the like being executed by, for example, the processing element 205. Thus, the databases, database instances, database management systems, data, applications, programs, program modules, code (source code, object code, byte code, compiled code, interpreted code, machine code) that embodies one or more computer functions described herein, executable instructions, and/or the like may be used to control certain aspects of the operation of the computing entity 200 with the assistance of the processing element 205 and operating system.

As indicated, in some embodiments, the computing entity 200 may also include one or more network interfaces 220 for communicating between various computing entities such as by communicating data, code, content, information, and/or similar terms used herein interchangeably that may be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. In some embodiments, the computing entity 200 communicates with another computing entity for uploading or downloading data or code. Similarly, the computing entity 200 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1X (1xRTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the computing entity 200 may include, or be in communication with, one or more input elements, such as a keyboard input, a mouse input, a touch screen/display input, motion input, movement input, audio input, pointing device input, joystick input, keypad input, and/or the like. The computing entity 200 may also include, or be in communication with, one or more output elements (not shown), such as audio output, video output, screen/display output, motion output, movement output, and/or the like.

### B. Example External Computing Entity

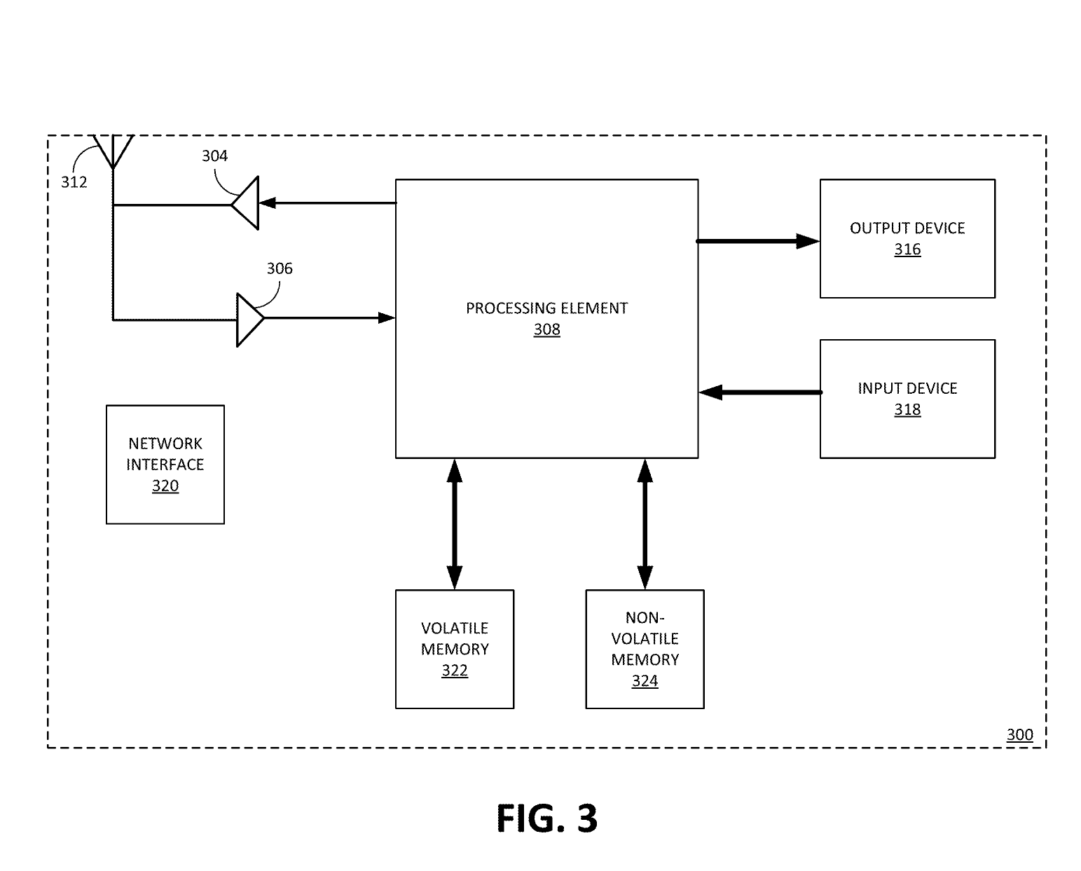

FIG. 3 provides an example computing entity 300 in accordance with some embodiments of the present disclosure. In general, FIG. 3 may be representative of components of any of the modules/systems described herein generally enabling processing within each component and communication between the components. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. As shown in FIG. 3, the computing entity 300 may include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly. Generally, computing entity 300 may be an example of the frontend component 120 of FIG. 1.

The signals provided to and received from the transmitter 304 and the receiver 306, correspondingly, may include signaling information/data in accordance with air interface standards of applicable wireless systems. In this regard, the computing entity 300 may be capable of operating with one or more air interface standards, communication protocols, modulation types, and access types. More particularly, the computing entity 300 may operate in accordance with any of a number of wireless communication standards and protocols, such as those described above with regard to the computing entity 200. In some embodiments, the computing entity 300 may operate in accordance with multiple wireless communication standards and protocols, such as UMTS, CDMA2000, 1xRTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the computing entity 300 may operate in accordance with multiple wired communication standards and protocols, such as those described above with regard to the computing entity 200 via a network interface 320.

Via these communication standards and protocols, the computing entity 300 may communicate with various other entities using mechanisms such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The computing entity 300 may also download code, changes, add-ons, and updates, for instance, to its firmware, software (e.g., including executable instructions, applications, program modules), and operating system.

The computing entity 300 may also comprise a user interface (that may include an output device 316 (e.g., display, speaker, tactile instrument, etc.) coupled to a processing element 308) and/or a user input interface (coupled to a processing element 308). For example, the user interface may be a user application, browser, user interface, and/or similar words used herein interchangeably executing on and/or accessible via the computing entity 300 to interact with and/or cause display of information/data from the computing entity 200, as described herein. The user input interface may comprise any of a plurality of input devices 318 (or interfaces) allowing the computing entity 300 to receive code and/or data, such as a keypad (hard or soft), a touch display, voice/speech or motion interfaces, or other input device. In some embodiments including a keypad, the keypad may include (or cause display of) the conventional numeric (0-9) and related keys (#, *), and other keys used for operating the computing entity 300 and may include a full set of alphabetic keys or set of keys that may be activated to provide a full set of alphanumeric keys. In addition to providing input, the user input interface may be used, for example, to activate or deactivate certain functions, such as screen savers and/or sleep modes.

The computing entity 300 may also include volatile memory 322 and/or non-volatile memory 324, which may be embedded and/or may be removable. For example, the non-volatile memory 324 may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory 322 may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile memory may store databases, database instances, database management systems, data, applications, programs, program modules, scripts, code (source code, object code, byte code, compiled code, interpreted code, machine code, etc.) that embodies one or more computer functions described herein, executable instructions, and/or the like to implement the functions of the computing entity 300. As indicated, this may include a user application that is resident on the computing entity 300 or accessible through a browser or other user interface for communicating with the computing entity 200 and/or various other computing entities.

In another embodiment, the computing entity 300 may include one or more components or functionalities that are the same or similar to those of the computing entity 200, as described in greater detail above. As will be recognized, these architectures and descriptions are provided for example purposes only and are not limited to the various embodiments.

### C. IMMUNODOM Visualization Tool

IMMUNODOM is a visualization tool developed to support the identification and analysis of high-affinity, disease-relevant peptide-HLA interactions, which are of particular interest to researchers focused on autoimmunity, infectious diseases, and cancer. By utilizing data from the Immune Epitope Database (IEDB), IMMUNODOM offers a streamlined platform for visualizing and comparing peptide-binding predictions across various HLA allotypes, which is key to understanding T cell recognition mechanisms. The IMMUNODOM visualization tool simplifies identification of immunodominant peptides by facilitating the automated plotting and overlay of binding data, thus helping researchers efficiently evaluate interactions between peptides and HLA molecules. IMMUNODOM enables the generation of publication-quality figures and direct analysis within a unified environment, potentially aiding in the identification of therapeutic targets and elucidating T cell responses.

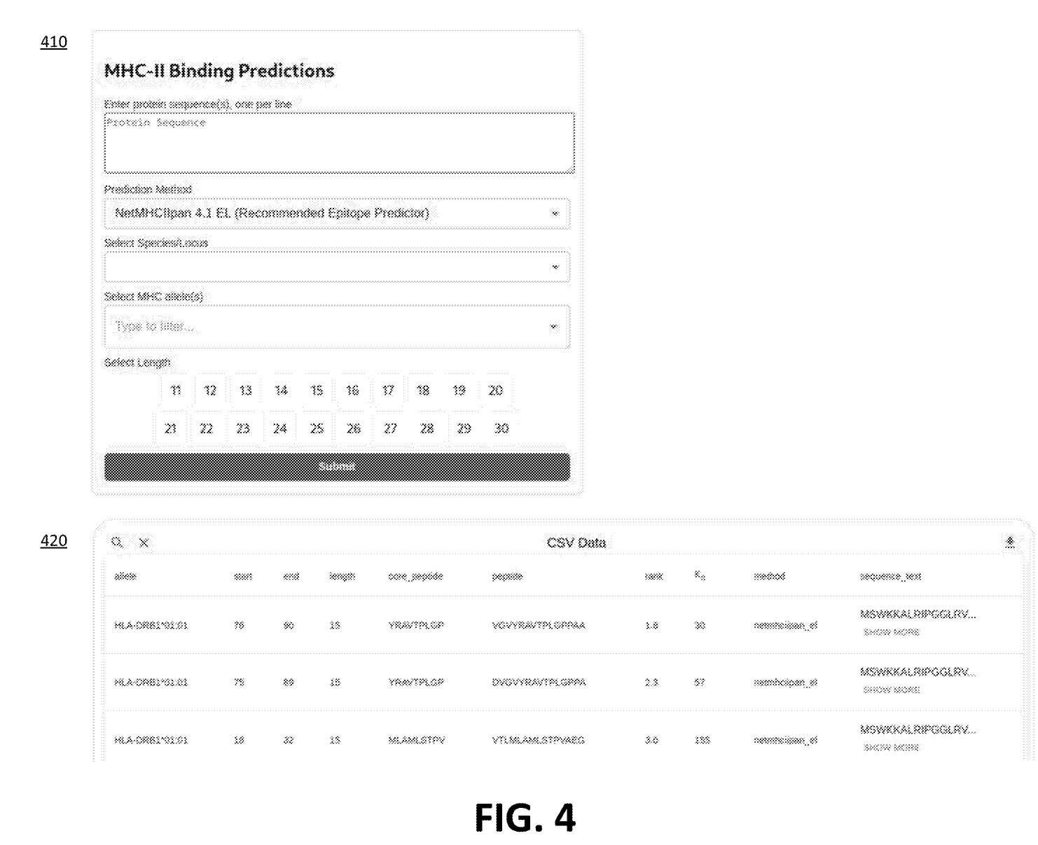

FIG. 4 depicts an example conservation of IEDB prediction tool functions in a subject web application in accordance with at least some embodiments of the present invention. As depicted, panel 410 illustrates the replication of the MHC-II Binding Prediction Tool query form within a subject web application, preserving customization options as available within the original IEDB prediction tool. Panel 420 illustrates how the subject web application facilitates access to text and numerical outputs from the prediction tool, thereby enabling both real-time analysis and data download.

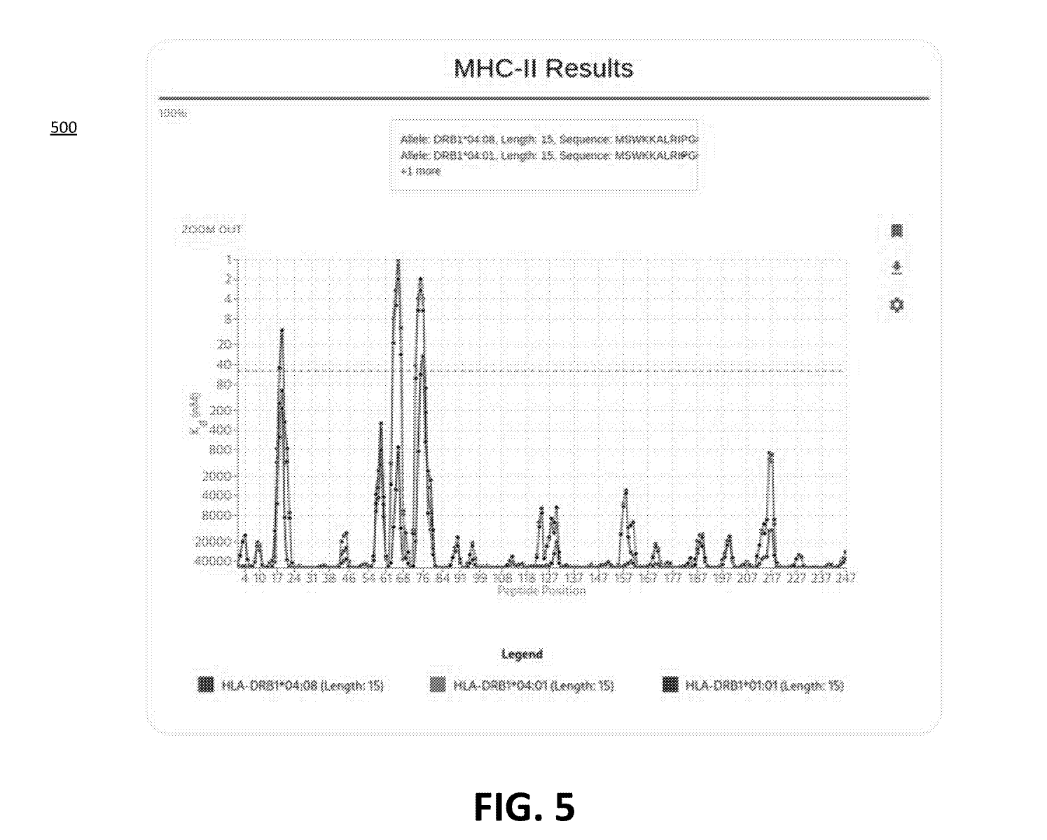

FIG. 5 depicts an example automated graphing 500 of T cell epitope predictions in accordance with at least some embodiments of the present invention. As depicted, the example automated graphing 500 depicts an automated plot of predicted T cell epitopes for a protein sequence queried against three HLA Class II allotypes. Embodiments of the present disclosure facilitate the direct visualization of prediction data, enabling users to assess the relative binding strengths across different allotypes within a single interface. Plots are customizable and can be downloaded for further analysis or presentation purposes.

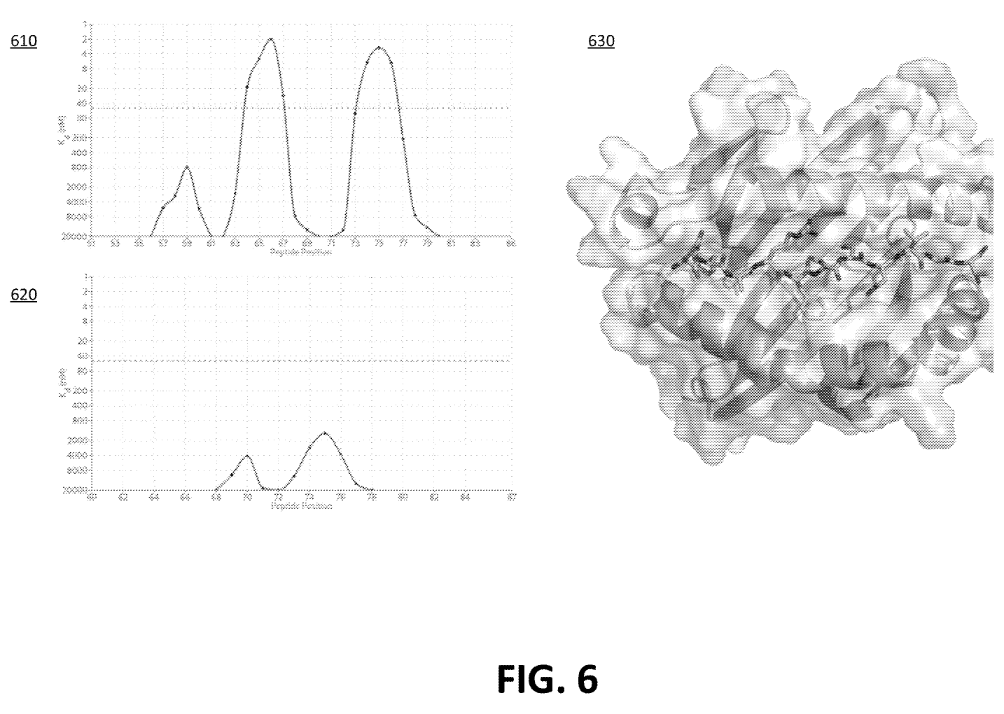

FIG. 6 depicts an example series of panels depicting identification of immunodominant epitopes in candidate autoantigens in accordance with at least some embodiments of the present invention. Analysis is shown of the HLA-DQB1*03:02 protein sequence to identify high-affinity peptide epitopes that bind to HLA molecules associated with Type 1 diabetes (T1D). Visualizations highlight epitopes with the strongest predicted binding affinities to HLA-DR4, which is linked to increased disease risk as depicted in panel 610. Visualizations highlight epitopes with the strongest predicted binding affinities to HLA-DR6, associated with disease protection as depicted in panel 620. The peptide with the highest affinity for HLA-DR4, REEYARFDSDVGVYR (positions 66-80 of DQB1*03:02), is shown in a structural model complexed with HLA-DR4. A dashed line indicates the threshold for high-affinity binding, set at a dissociation constant (Kd) of 50 nM or lower. The REEYARFDSDVGVYR peptide is shown as sticks, the HLA-DR alpha-chain is shown as a ribbon diagram and molecular surface at the top half of panel 630, and the HLA-DR beta-chain (HLA-DRB1*04:01) is shown as a ribbon diagram and molecular surface at the bottom half of panel 630.

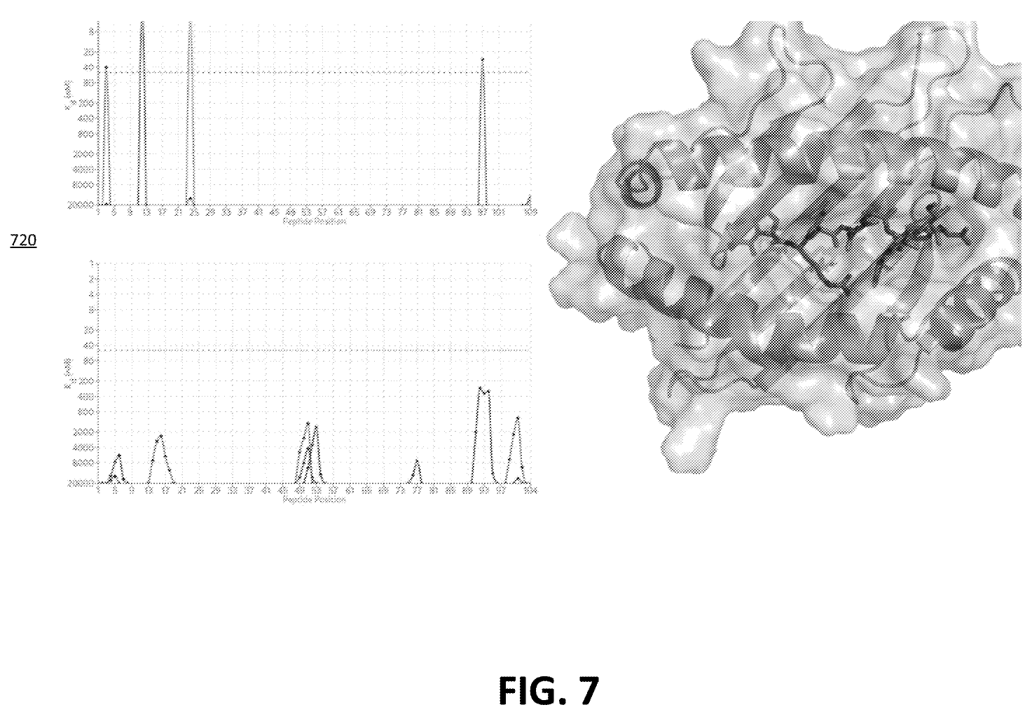

FIG. 7. depicts an example series of panels depicting identification of immunodominant epitopes in MART-1 in accordance with at least some embodiments of the present invention. Analysis is shown of the MART-1 tumor-associated antigen to identify high-affinity peptides that bind to both Class I and Class II HLA allotypes. Panel 710 shows peptides predicted to have strong affinity for the common Class I HLA-B alleles, including HLA-B*35:01, HLA-B*40:01, HLA-B*44:03, and HLA-B*07:02. Panel 720 depicts peptides likely to bind to common Class II HLA-DR alleles, such as HLA-DRB1*15:01, DRB1*07:01, and DRB1*03:01. Panel 730 shows the MART-1 peptide MPREDAHFIY (positions 1-10), which has the highest predicted affinity for HLA-B*35:01 is shown as sticks, whereas HLA-B*35:01 is shown as a ribbon diagram and molecular surface.

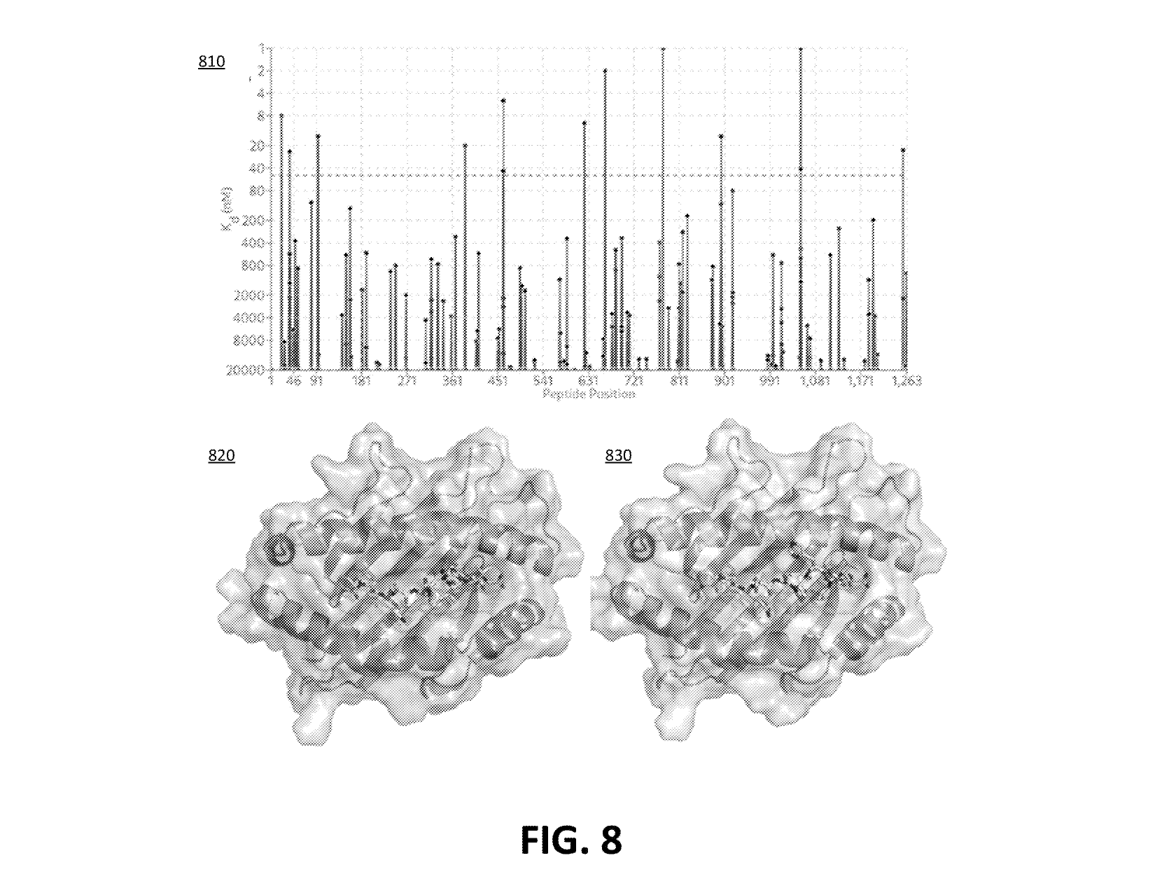

FIG. 8 depicts an example series of panels depicting identification of high-affinity T cell epitopes from SARS-CoV-2 spike protein in accordance with at least some embodiments of the present invention. Analysis is shown of the spike protein sequence from SARS-CoV-2 aimed at identifying high-affinity peptides predicted to bind to common HLA class I allotypes. Panel 810 shows peptides with the highest predicted affinity for common HLA-B alleles, including HLA-B*35:01, B*15:01, B*40:01, B*44:03, B*07:02, B*51:01, B*40:02, B*07:04, B*58:01, and B*08:01. The peptide FPQSAPHGVVF (positions 1052-1062), which has the highest predicted affinity, is highlighted. Panel 820 shows the FPQSAPHGVVF peptide in a structural model complexed with HLA-B*35:01, with an estimated dissociation constant (Kd) of 1 nM. Panel 830 shows the FPQSAPHGVVF peptide complexed with HLA-B*07:02, with a predicted high affinity Kd value of 41 nM. The FPQSAPHGVVF peptide is shown as sticks, HLA-B*35:01 is shown as a ribbon diagram and molecular surface in panel 820, and the HLA-B*07:02 is shown as a ribbon diagram and molecular surface in panel 830.

### IV. Examples of Certain Terms

In some embodiments, the terms “user system,” “user device,” “user computing device,” “user computing entity,” and the like may refer to any system configured to run a drug discovery and optimization platform as described herein. In general, the user system may refer to any system capable of providing an interface to a user device via which a user can interact with a drug discovery application.

In some embodiments, the terms “Radiomics and Augmented Intelligence Laboratory,” “Radiomics-based AI models,” and the like refer to machine learning models configured to identify biomarkers, enable precise tumor characterization and treatment response evaluation, and support personalized therapeutic strategies.

In some embodiments, the terms “Computational Renal Pathology Suite,” “ComPRePS,” and the like refer to a cloud-based image analysis platform configured to integrate advanced deep neural network-based AI technologies to automate the analysis of high-resolution whole-slide images of histologically stained tissue sections.

In some embodiments, the terms “Radiology Pathology Image Alignment,” “RAPHIA,” and the like refer to an advanced deep learning pipeline designed to facilitate the precise registration of magnetic resonance imaging with whole-mount histopathology images of the prostate.

In some embodiments, the terms “Electroencephalography Source Imagine with Convolutional Neural Networks,” “ESI/CNN,” and the like refer to machine learning models configured to classify motor imagery tasks to enhance the decoding of neural signals associated with motor functions, useful for developing brain-computer interfaces and neuroprosthetics.

In some embodiments, the term “miRomics” refers to a machine learning approach to analyze the unique miRomics expression profiles, thereby enabling a deeper understanding of disease progression and biomarkers.

In some embodiments, the terms “Functional Unit State Identification in Whole Slide Images,” “FUSION,” and the like refer to a tool that provides users with a broad array of visualization and analytical tools including deep learning-based algorithms for in-depth interrogation of spatial-OMICS datasets and their associated high-resolution histology images.

In some embodiments, the term “Machine Gun Docking” refers to a high throughput deep docking acceleration of structure-based virtual screening.

In some embodiments, the term “DyScore” refers to a bosting scoring method with dynamic properties for identifying true binders and nonbinders in structure-based drug discovery.

In some embodiments, the terms “regulatory-ready data component,” “regulatory-ready data system,” “regulatory-ready data application,” “validation system,” “validation component,” and the like refer to components, systems, or applications configured to manage and process data in a manner that meets requirements of specific regulations and standards. Regulatory-ready data components may be configured to ensure data accuracy, security, and compliance with privacy laws, industry regulations, and internal policies, for example. Regulatory-ready data components may be configured to maintain data accuracy and integrity, data security, data privacy, data compliance, data governance, reporting and auditing, and the like. In at least some embodiments, regulatory-ready data components are trained or conditioned using an ingested set of compliance guidelines.

In some embodiments, the terms “screening techniques,” “parallel screening techniques,” “real-time screening techniques,” “real-time parallel screening techniques,” and the like refer to techniques for filtering or otherwise removing unimportant variables from a dataset, thereby making subsequent analysis more efficient and targeted. Screening techniques may enable identification of relevant variables with respect to omics data with a reduced computational burden. Appropriate screening techniques may include, but are not limited to, clustering-based screening, statistical screening, penalized regression, multi-omics integration, omics generative adversarial networks,

### V. Example System Operations

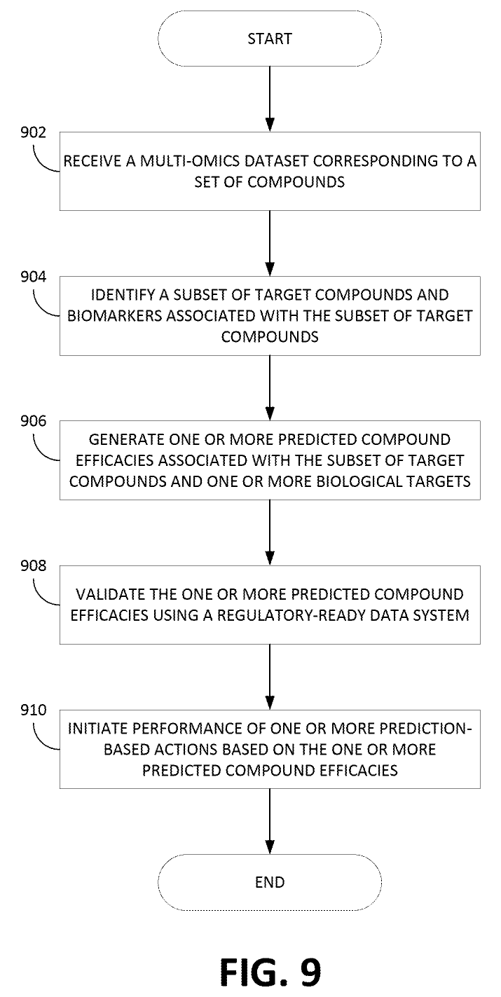

FIG. 9 depicts an example process 900 for identifying and optimizing drug candidates. As depicted, the process 900 includes receiving (902) a multi-omics dataset corresponding to a set of compounds, identifying (904) a subset of target compounds and biomarkers associated with the subset of target compounds, generating (906) one or more predicted compound efficacies associated with the subset of target compounds and a set of biological targets, validating (908) the one or more predicted compound efficacies using a validation component, and causing (910) execution of one or more prediction-based actions based on the one or more predicted compound efficacies. Example process 900 may enable drug candidate optimization relative to a set of biological targets.

At step 902, framework 100 is configured to receive a multi-omics dataset corresponding to a set of compounds. Receiving (902) a multi-omics dataset corresponding to a set of compounds may include receiving a dataset comprising data integrated from multiple omics sources, such as genomics, transcriptomics, proteomics, and metabolomics to gain a more comprehensive understanding of biological compounds and/or systems. In at least some embodiments, receiving (902) a multi-omics dataset includes receiving a dataset corresponding to a selected set of compounds.

In some embodiments, the multi-omics dataset comprises genomics, transcriptomics, and proteomics data integrated for comprehensive target identification and drug candidate evaluation. The multi-omics dataset may comprise genomics data including genetic variations and mutations associated with diseases, providing targets for novel drug therapies. The genomics data may be obtained by leveraging advanced genomic technologies including single-cell genomics and high-throughput sequencing services that enable detailed genetic profiling of individual cells. This approach may enable identifying unique genetic markers and understanding disease mechanisms at an unprecedented resolution.

The multi-omics dataset may further comprise transcriptomics data charting changes in gene expression across different conditions and responses to treatments. The transcriptomics data may enable identification of biomarkers and therapeutic targets by analyzing differential gene expression patterns associated with disease states and compound responses.

In some embodiments, the multi-omics dataset additionally comprises proteomics data obtained from mass spectrometry and other high-throughput technologies analyzing protein expressions and interactions. The proteomics data is useful for understanding disease mechanisms at a molecular level and for validating drug targets within the set of compounds.

The integration of genomics, transcriptomics, and proteomics data within the multi-omics dataset facilitates a comprehensive approach to drug discovery. This multi-omics integration supports the initial discovery of drug targets through optimization and validation of drug candidates, increasing accuracy and enabling personalized therapeutic solutions for the set of compounds.

In some embodiments, an AI-driven tool (e.g., AIGen) is configured to analyze the multi-omics dataset using deep learning and statistical methods to process large-scale multi-omics data including genetics, single-cell genomics, and metagenomics. The AI-driven tool enables the one or more processors to extract insights from the multi-omics dataset for identifying the subset of target compounds associated with one or more biomarkers of a biological target.

At step 904, framework 100 is configured to identify a subset of target compounds and biomarkers associated with the subset of target compounds/target subjects. Identifying (904) a subset of target compounds and biomarkers associated with the subset of target compounds may include leveraging AI tools to extract and analyze quantitative features from medical images to identify biomarkers for a range of diseases. In general, identifying (904) a subset of target compounds and biomarkers associated with the subset of target compounds may include identifying one or more biomarkers associated with a target subject, and subsequently identifying a subset of target compounds associated with the identified biomarkers.

In some embodiments, identifying (904) a subset of target compounds and biomarkers associated with the subset of target compounds/target subjects comprises using real-time parallel screening techniques based at least in part on the multi-omics dataset. The real-time parallel screening techniques may enable simultaneous evaluation of thousands of compounds across multiple biological targets, facilitating rapid identification of promising therapeutic candidates.

The real-time parallel screening techniques may utilize advanced robotic systems and microtiter plates with 1,536 wells for high-density screening formats. This parallel screening technology enables the simultaneous testing of thousands of molecules across multiple biological targets, significantly enhancing the scope and speed of compound identification. The high-throughput screening subsystem may efficiently translate benchtop assays into high-density formats suitable for large-scale screening, accelerating the identification of potential therapeutic leads by allowing rapid assessment of compound libraries against diverse pharmacological targets.

In some embodiments, identifying the one or more biomarkers comprises genomic profiling to identify genetic variations and mutations associated with diseases, providing targets for novel drug therapies. The genomic profiling may leverage single-cell genomics and high-throughput sequencing services that enable detailed genetic profiling of individual cells. This approach is instrumental in identifying unique genetic markers and understanding disease mechanisms at an unprecedented resolution, enabling the identification of biomarkers associated with the subset of target compounds.

Identifying the one or more biomarkers may comprise proteomic profiling employing mass spectrometry and high-throughput technologies to identify molecular signatures that can predict drug efficacy or the likelihood of adverse reactions to treatments. The proteomic profiling analyzes protein expressions and interactions, which inform understanding of disease mechanisms at a molecular level and validation of drug targets associated with the subset of target compounds.

The one or more biomarkers may comprise predictive biomarkers configured to guide patient selection or track therapeutic response. Genomic and proteomic profiling may be employed to identify molecular signatures that can predict drug efficacy or the likelihood of adverse reactions to treatments. The predictive biomarkers are integral to personalizing patient care, ensuring that therapies are tailored to individual genetic profiles and physiological characteristics, which increases the chances of successful treatment outcomes.

The one or more biomarkers may further comprise translational biomarkers configured to bridge preclinical models and clinical applications. The translational biomarkers include biomarkers for Alzheimer’s disease that help in identifying early stages of the disease by detecting specific proteins or genetic markers, which can be targeted for early therapeutic intervention.

In some embodiments, the one or more biomarkers additionally comprise cancer biomarkers involving identifying genetic expressions and mutations specific to various cancer types. These cancer biomarkers facilitate targeted therapy regimes in clinical trials, improving the precision of treatments tailored to individual genetic profiles. Such discoveries not only expedite the process from lab research to practical treatment options but also enhance the efficacy and personalization of cancer therapies for the subset of target compounds.

Identifying the subset of target compounds may further comprise identifying immunodominant peptide epitopes for any given Human Leukocyte Antigen (HLA) allotype. The process 900 may enable precise identification of immunodominant peptide epitopes, allowing researchers to predict and manipulate immune responses more effectively.

At step 906, framework 100 is configured to generate one or more predicted compound efficacies associated with the subset of target compounds and the set of biological targets. Generating (906) one or more predicted compound efficacies associated with the subset of target compounds and the set of biological targets may include predicting a treatment efficacy associated with application of the subset of target compounds with respect to the set of biological targets. In general, generating (906) one or more predicted compound efficacies includes predicting a likelihood of a successful outcome for using the subset of target compounds to treat the set of biological targets.

In some embodiments, the method comprises generating (906) one or more predicted compound efficacies comprises forecasting compound efficacy, toxicity, or pharmacokinetics, enabling rapid evaluation of therapeutic potential for the subset of target compounds. In some embodiments, generating (906) the one or more predicted compound efficacies comprises utilizing DyScore, an innovative scoring method that utilizes dynamic properties to enhance the identification of true binders and non-binders in protein-ligand interactions. DyScore incorporates a nuanced consideration of the dynamic nature of molecular interactions, leading to more accurate predictions of binding affinities which are useful for identifying promising drug candidates early in the drug discovery process. Unlike traditional scoring functions that often suffer from inaccuracies due to their simplification of complex molecular interactions, DyScore provides a more reliable tool for evaluating the potential effectiveness of molecules within the subset of target compounds.

In some embodiments, generating (906) the one or more predicted compound efficacies comprises utilizing the Machine Gun Docking approach, which screens molecular fragments against multiple target sites on the biological target. The Machine Gun Docking approach achieves an approximately 80% hit rate for identifying potent and versatile leads, attributed to the technique’s ability to rapidly and accurately screen a vast array of molecular fragments against multiple structural pockets. This structure-based drug design approach leverages multiple structural pockets to identify and select potential lead compounds from the subset of target compounds.

In some embodiments, generating (906) the one or more predicted compound efficacies comprises performing detailed simulations that predict the binding affinity and stability of drug candidates with their target molecules. The molecular dynamics simulations enable analysis of a vast number of molecules in a reduced time frame, ensuring that potential drug candidates can be quickly assessed and refined based on their molecular interactions.

In some embodiments, generating (906) the one or more predicted compound efficacies comprises performing quantitative structure-activity relationship (QSAR) modeling for optimizing binding affinity and selectivity of compounds within the subset of target compounds. Compatibility with advanced modeling and simulation software facilitates the detailed study of drug-target interactions at the molecular level, useful for optimizing binding affinity and selectivity. This integration enables researchers to leverage cutting-edge computational tools for structure-based drug design and virtual screening of the subset of target compounds.

In some embodiments, generating (906) the one or more predicted compound efficacies comprises utilizing IMMUNODOM to predict immunodominant peptide epitopes for HLA allotypes. IMMUNODOM generates binding affinity predictions expressed as dissociation constant (Kd) values, classifying epitope candidates as high affinity, moderate affinity, or low affinity.

In some embodiments, generating (906) the one or more predicted compound efficacies comprises AI-based generation of novel molecular structures optimized for specific targets based on predicted compound efficacy. De Novo drug design utilizes the one or more machine learning models to generate novel molecular structures that are optimized for binding to the biological target. The AI-based generation enables the creation of novel therapeutic candidates within the subset of target compounds that exhibit predicted high efficacy against the biological target.

At step 908, framework 100 is configured to validate the one or more predicted compound efficacies using a regulatory-ready data component. Validating (908) the one or more predicted compound efficacies using a validation component may include validating the predicted compound efficacy according to one or more defined guidelines for treatment or clinical testing. Validating (908) the one or more predicted compound efficacies may include administering a treatment according to the one or more predicted compound efficacies and determining whether the treatment yields results as indicated by the predicted compound efficacy. In general, validating (908) the one or more predicted compound efficacies includes validating that administration of the compound complies with a selected set of guidelines, and, in an instance where treatment is administered accordingly, determining whether the treatment yields results as expected according to the predicted compound efficacy.

In some embodiments, validating (908) the one or more predicted compound efficacies associated with the subset of target compounds and the biological target comprises preclinical validation using phenotypic screening with organoids and 3D cultures configured to mimic in vivo conditions, enabling assessment of therapeutic potential prior to clinical development.

Validating (908) the one or more predicted compound efficacies may comprise phenotypic screening using functional assays to identify compounds that affect cellular or disease phenotypes. The phenotypic screening enables researchers to observe how compounds affect disease phenotypes directly, facilitating the discovery of novel therapeutics by linking drug effects to observable outcomes without prior knowledge of the drug’s mechanism. This approach allows for validation of predicted compound efficacy across a broad spectrum of both common and rare diseases.

Validating (908) the one or more predicted compound efficacies further comprises utilizing organoids and 3D cultures to mimic in vivo conditions for compound evaluation. Organoids derived from patient tumor samples may be used to test the efficacy of therapeutic agents, allowing for a personalized medicine approach that predicts individual responses to treatment before clinical application. Brain organoids may be developed to model neurodegenerative conditions such as Alzheimer’s disease, providing insights into pathogenesis and serving as platforms for testing potential neuroprotective compounds. Intestinal organoids facilitate the study of host-pathogen interactions in infectious disease research, enabling screening for antiviral compounds that could inhibit viral replication within human tissues.

Validating (908) the one or more predicted compound efficacies additionally comprises biophysical validation methods including X-ray crystallography, cryo-electron microscopy (cryo-EM), and surface plasmon resonance (SPR) for target validation and hit optimization. X-ray crystallography and cryo-EM allow for detailed structural analysis of drug-target interactions, providing insights into the molecular interactions and dynamics useful for effective drug design. SPR provides real-time measurement of binding kinetics and affinity between compounds and their targets, enabling validation of predicted binding affinities generated by the one or more machine learning models.

Validating (908) the one or more predicted compound efficacies further comprises preclinical validation in animal models with proven ability to produce candidates with high success rates. The validation component may be configured to perform in vivo efficacy testing and in vivo toxicology testing to assess therapeutic potential and safety profiles of the subset of target compounds. This preclinical validation ensures that compounds demonstrating predicted efficacy in silico and in vitro translate to effective therapeutic agents in living systems.

Validating (908) the one or more predicted compound efficacies may further comprise hit-to-lead conversion validation with high hit rates and strong leads for further development. In some embodiments, validating (908) the one or more predicted compound efficacies further comprises pharmacokinetic and pharmacodynamic validation through rapid optimization of pharmacokinetics, pharmacodynamics, and drug-like properties. The validation component may be configured to perform preliminary ADME (absorption, distribution, metabolism, and excretion) studies and detailed PK assessments to ensure that compounds with predicted efficacy exhibit favorable drug-like characteristics for therapeutic development.

Validating (908) the one or more predicted compound efficacies may additionally comprise translational validation using biomarkers to bridge preclinical models and clinical applications. The use of biomarkers connects preclinical validation results with clinical applicability, ensuring that validated compounds demonstrate therapeutic potential that translates from laboratory settings to patient care. This translational validation approach expedites the process from lab research to practical treatment options while enhancing the efficacy and personalization of therapies for the biological target.

At step 910, framework 100 is configured to initiate performance of one or more prediction-based actions corresponding to the one or more predicted compound efficacies. The prediction-based actions may enable identification and analysis of immunodominant peptide epitopes for therapeutic development across multiple disease areas including autoimmunity, cancer immunotherapy, and infectious disease vaccine development.

In some embodiments, the prediction-based actions comprise identifying immunodominant peptide epitopes from user-supplied protein sequences. The framework 100 may be configured to generate downloadable text and numerical data including ranked lists of predicted binding core sequences and scores based on predicted binding affinity scores, thereby enabling researchers to efficiently evaluate interactions between peptides and HLA molecules for therapeutic target identification.

The prediction-based actions may further comprise classifying binding affinity of identified epitopes. The framework 100 may be configured to identify high affinity, moderate affinity, and low affinity binding T cell epitope candidates based on predicted affinity constants expressed as dissociation constant (Kd) values.

In some embodiments, the prediction-based actions comprise comparing peptide interactions across HLA allotypes. The framework 100 may be configured to analyze protein sequences to identify peptides predicted to bind to risk-associated HLA alleles versus protective HLA alleles, enabling comparison of binding affinities across different allotypes. This comparative analysis facilitates identification of epitopes associated with differential disease risk or protection.

The prediction-based actions may comprise identifying tumor-associated antigen epitopes for immunotherapy applications. The framework 100 may be configured to identify T cell epitopes from tumor-associated antigens that are likely to bind to multiple common HLA allotypes, enabling stimulation of T cells to attack and destroy cancer cells in a broad patient population.

In some embodiments, the prediction-based actions comprise identifying viral epitopes for vaccine development. The system is configured to identify sequences from viral proteins that bind to multiple common class I HLA molecules to enhance vaccine development. The framework 100 may enable incorporation of key epitopes into vaccines to provide robust protection while excluding sequences known to cause complications.

The prediction-based actions may comprise visualizing epitope predictions across multiple HLA allotypes. The framework 100 may be configured to provide visualization through color coding for rapid identification of immunodominant epitopes in context of multiple specific HLA allotypes within a single analysis session.

In some embodiments, the prediction-based actions comprise generating structural models of peptide-HLA complexes. The framework may be configured to generate structural models using TCRmodel to visualize predicted high-affinity peptide-HLA interactions, providing molecular-level insight into the binding interactions between identified epitopes and their respective HLA molecules.

The prediction-based actions may comprise guiding personalized immunotherapy strategies. The framework 100 may be configured to analyze tumor-associated antigens to identify epitopes that could be targeted by T cells, aiding in the design of cancer vaccines and personalized immunotherapy strategies tailored to individual patient HLA profiles.

In at least some embodiments, the prediction-based actions may include automated alerts, automated instructions to insulin delivery devices, automated adjustments to medical equipment, automated adjustments to allocations of medical, computing, hospital, facility, and/or human resources. Further, such automated actions may include automated physician notification actions, automated patient notification actions, automated appointment scheduling actions, automated prescription recommendation actions, automated drug prescription generation actions, automated implementation of precautionary actions, automated record updating actions, automated datastore updating actions, automated hospital preparation actions, automated workforce management operational management actions, automated server load balancing actions, automated resource allocation actions, automated call center preparation actions, automated hospital preparation actions, automated pricing actions, automated plan update actions, automated alert generation actions, generating a diagnostic report, generating action scripts, generating one or more electronic communications, and/or the like. The one or more prediction-based actions may further include displaying visual renderings of the aforementioned examples of prediction-based actions in addition to values, charts, and representations associated with the one or more policy scores and the prediction output using a prediction output user interface.

In at least some embodiments, framework 100 may additionally be configured to obtain structural information for a target protein, identify two or more distinct druggable structural pockets on said protein, apply artificial intelligence algorithms to model ligand-binding potential for each of the distinct druggable structural pockets, conduct high-throughput in silico screening against each of the identified pockets using a library of small molecules, and select candidate compounds that exhibit predicted high binding affinity to one or more of the pockets. In at least some embodiments, the structural information is derived from X-ray crystallography, cryo-electron microscopy, or AlphaFold-predicted models. In at least some embodiments, the artificial intelligence algorithms comprise a deep learning model trained to predict binding affinities, pocket druggability, or conformational flexibility.

In at least some embodiments, the high throughput in silico screening is performed using molecular docking, molecular dynamics (MD) simulations, or free energy perturbation calculations. In at least some embodiments, framework 100 is additionally configured to rank the identified pockets by predicted druggability and select compounds targeting the top-ranked pockets for downstream validation. In at least some embodiments, framework 100 is additionally configured to synthesize or acquire the selected compounds, and subject them to one or more experimental validation assays. Such validation assays include, but are not limited to, biophysical binding assays, enzymatic activity assays, or cell-based functional assays. In at least some embodiments, identified synergistic compounds bind to distinct pockets on the same protein or protein complex, thereby enhancing overall efficacy. In at least some embodiments, compounds binding different pockets are designed and synthesized for use in combination therapy to modulate protein function with enhanced specificity or reduced resistance. In at least some embodiments, the identified pockets include allosteric sites and protein-protein interaction surfaces. In at least some embodiments, framework 100 is further configured to integrate omics data or disease-specific mutations to prioritize pocket selection and compound optimization.

In at least some embodiments, the process 900 may be executed iteratively (e.g., to incorporate multiple experimental validation assays. In doing so, iterative execution of the process 900 may enable iterative improvement of drug candidate specificity, drug safety, and drug solubility.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method comprising:
receiving, by one or more processors, a multi-omics dataset corresponding to a set of compounds;
identifying, by the one or more processors and using real-time parallel screening techniques, and based at least in part on the multi-omics dataset, a subset of target compounds from the set of compounds associated with one or more biomarkers of a biological target;
generating, by the one or more processors and using one or more machine learning models, one or more predicted compound efficacies associated with the subset of target compounds and the biological target;
validating, by the one or more processors, the one or more predicted compound efficacies associated with the subset of target compounds and the biological target; and
initiating, by the one or more processors and based on the one or more predicted compound efficacies, performance of one or more prediction-based actions.

2. The computer-implemented method of claim 1, wherein one or more components for validating the one or more predicted compound efficacies provide efficient hit-to-lead conversion.

3. The computer-implemented method of claim 1, further comprising predicting, by the one or more processors, compound toxicity and pharmacokinetics associated with the subset of target compounds and the biological target.

4. The computer-implemented method of claim 1, wherein the real-time parallel screening techniques include phenotypic screening techniques for analyzing the set of compounds and the multi-omics dataset.

5. The computer-implemented method of claim 1, wherein the real-time parallel screening techniques include biophysical screening techniques for analyzing the set of compounds and the multi-omics dataset.

6. The computer-implemented method of claim 1, further comprising enabling, by the one or more processors, de novo molecular design based, at least in part, on the one or more predicted compound efficacies and the subset of target compounds.

7. The computer-implemented method of claim 1, wherein validating the one or more predicted compound efficacies comprises verifying compliance with one or more regulatory documents.

8. A drug discovery platform comprising:
a structural analysis module configured to identify one or more druggable pockets on a target protein;
an artificial intelligence module configured to (i) prioritize the one or more druggable pockets and (ii) predict one or more ligand interactions;
a screening engine configured to perform virtual screening on the one or more druggable pockets simultaneously; and
a validation engine configured to evaluate a set of candidate compounds to determine one or more binding affinities associated with the set of candidate compounds and the one or more druggable pockets.

9. The drug discovery platform of claim 8, wherein the structural analysis module is further configured to identify one or more cryptic pockets that are not observable in static conformations.

10. The drug discovery platform of claim 9, wherein the structural analysis module is further configured to use ensemble-based or MD-derived structures to identify the one or more cryptic pockets.

11. The drug discovery platform of claim 8, wherein the artificial intelligence module is further configured to incorporate reinforcement learning to refine compound evaluation based at least in part on one or more experimental results.

12. The drug discovery platform of claim 8, wherein the structural analysis module is further configured to derive structural information from X-ray crystallography, cryo-electron microscopy, or AlphaFold-predicted models.

13. The drug discovery platform of claim 8, wherein the artificial intelligence module is further configured to predict binding affinities, pocket druggability, or conformational flexibility using a deep learning model.

14. The drug discovery platform of claim 8, wherein the validation engine is further configured to (i) determine a ranking of the one or more druggable pockets based at least in part on one or more predicted druggability values associated with the one or more druggable pockets, and (ii) select one or more compounds of the set of candidate compounds based at least in part on the ranking of the one or more druggable pockets.

15. The drug discovery platform of claim 14, further comprising a compound synthesis module configured to (i) synthesize the selected one or more compounds and (ii) subject the selected one or more compounds to one or more experimental assays.

16. One or more non-transitory computer-readable storage media including instructions that, when executed by one or more processors, cause the one or more processors to perform operations comprising:
receiving a multi-omics dataset corresponding to a set of compounds;
identifying, using real-time parallel screening techniques, and based at least in part on the multi-omics dataset, a subset of target compounds from the set of compounds associated with one or more biomarkers of a biological target;
generating, using one or more machine learning models, one or more predicted compound efficacies associated with the subset of target compounds and the biological target;
validating the one or more predicted compound efficacies associated with the subset of target compounds and the biological target, and
initiating, based on the one or more predicted compound efficacies, performance of one or more prediction-based actions.

17. The one or more non-transitory computer-readable storage media of claim 16, wherein the real-time parallel screening techniques include phenotypic screening techniques for analyzing the set of compounds and the multi-omics dataset.

18. The one or more non-transitory computer-readable storage media of claim 16, wherein the real-time parallel screening techniques include biophysical screening techniques for analyzing the set of compounds and the multi-omics dataset.

19. The one or more non-transitory computer-readable storage media of claim 16, the operations further comprising enabling de novo molecular design based, at least in part, on the one or more predicted compound efficacies and the subset of target compounds.

20. The one or more non-transitory computer-readable storage media of claim 16, wherein validating the one or more predicted compound efficacies comprises verifying compliance with one or more regulatory documents.
